(19) United States
(12) Patent Application Publication
(10) Pub. No.: US 2016/0339206 A1
(43) Pub. Date: Nov. 24, 2016

(54) Delivering Therapeutics to Tissue and Related Systems and Devices

(71) Applicant: Atanse, Inc., Belmont, MA (US)

(72) Inventor: Miles G. Cunningham, Waltham, MA (US)

(21) Appl. No.: 15/158,499

(22) Filed: May 18, 2016

Related U.S. Application Data
(63) Continuation-in-part of application No. 13/699,464, filed on Nov. 21, 2012.
(60) Provisional application No. 61/348,064, filed on May 25, 2010.
(60) Provisional application No. 62/163,897, filed on May 19, 2015.

(51) Int. Cl. A61M 25/00

(52) U.S. Cl. CPC A61M 25/0074; A61M 2210/0693; A61M 2025/0002

(57) ABSTRACT

In some aspects, systems for delivering a therapeutic agent to a selected site in a subject can include a substantially rigid guide cannula defining an axial bore having an open proximal end and an opening near its distal end; and a delivery cannula configured to fit within the guide cannula axial bore, the delivery cannula being pre-formed in a non-straight predetermined shape that differs from a shape of the guide cannula axial bore.

### RELATED APPLICATIONS

This application is a continuation-in-part of U.S. Ser. No. 13/699,464 filed on Nov. 21, 2012 and titled “Systems and Methods for Delivering Therapeutic Agents to Selected Sites in a Subject,” which is a National Stage Entry of International Application Number PCT/US 11/37867, filed on May 25, 2011 and titled “Systems and Methods for Delivering Therapeutic Agents to Selected Sites in a Subject,” which claims priority to U.S. Provisional Application No. 61/348,064, filed May 25, 2010, the contents of all of which are hereby incorporated herein by reference in their entirety. This application also claims priority to U.S. Provisional Application No. 62/163,897, filed May 19, 2015, the contents of which are hereby incorporated herein by reference in their entirety.

### TECHNICAL FIELD

This disclosure relates generally to therapeutic delivery systems and more specifically to delivering therapeutics to brain tissue and to related systems and devices.

### BACKGROUND

As new therapeutics are being developed to treat a damaged and/or diseased Central Nervous System (CNS), a need to deliver them to discrete areas of the brain arises. In many cases, the therapeutic must be delivered to multiple target locations in the brain. Traditional systems, such as for the delivery of therapeutic stem cells, have used a straight, rigid cannula with a relatively large diameter, e.g., 0.9 mm or greater. In order to deliver a medicine to multiple locations, however, the delivery cannula typically makes multiple passes through brain tissue. With each penetration, the risk of surgical complications, such as, hemorrhage, edema, structural damage, etc., increases.

### SUMMARY

In some aspects, systems for delivering a therapeutic agent to a selected site in a subject can include a substantially rigid guide cannula defining an axial bore having an open proximal end and an opening near its distal end; and a delivery cannula configured to fit within the guide cannula axial bore, the delivery cannula being pre-formed in a non-straight predetermined shape that differs from a shape of the guide cannula axial bore.

Embodiments can include one or more of the following features.

In some embodiments, the non-straight predetermined shape of the delivery cannula causes a portion of the delivery cannula disposed within the guide cannula to be resiliently biased to conform to the shape of the guide cannula axial bore. In some cases, the portion of the delivery cannula disposed within the guide cannula is resiliently biased in a substantially straight orientation.

In some embodiments, a distal portion of the delivery cannula extending from the opening of the guide cannula resumes the non-straight predetermined shape. In some embodiments, the non-straight predetermined shape comprises a curved profile. In some embodiments, the non-straight predetermined shape comprises a three dimensional profile. In some embodiments, the non-straight predetermined shape comprises a spiral shape. In some embodiments, the non-straight predetermined shape comprises a bend of at least 5 degrees. In some embodiments, the non-straight predetermined shape comprises at least 360 degrees of total bend angle. In some embodiments, the at least 360 degrees of total bend are formed along a common plane. In some embodiments, the non-straight predetermined shape corresponds to an identified structure to be treated by the therapeutic. In some embodiments, the identified structure comprises a fiber tract. In some embodiments, the identified structure comprises a portion of tissue affected by a medical incident. In some embodiments, the distal portion of the delivery cannula comprises a step tapered region. In some embodiments, a ratio of a width of a larger portion of the step tapered region to a width of a smaller portion of the step tapered region is at least about 2:1. In some embodiments, the delivery cannula comprises a conductive portion forming an electrical circuit between a distal end of the delivery cannula and a proximal end of the delivery cannula. In some embodiments, the system also includes an insulating material disposed over a portion of the conductive portion. In some embodiments, the conductive portion comprises the delivery cannula being formed of a shape memory alloy.

In some aspects methods of delivering a therapeutic agent to a selected site in a subject can include: identifying a geometric property of an affected area to be treated with the therapeutic agent; causing formation of a non-straight predetermined shape in the delivery cannula, the non-straight predetermined shape being based on the geometric property of the affected area; and inserting the delivery cannula having the non-straight predetermined shape into a substantially rigid guide cannula defining an axial bore having an open proximal end and an opening near its distal end.

Embodiments can include one or more of the following features.

In some embodiments, methods can also include, upon insertion of the delivery cannula into the guide cannula, resiliently biasing a portion of the delivery cannula disposed within the guide cannula to conform to the guide cannula axial bore. In some embodiments, methods can also include inserting the delivery cannula further into guide cannula thereby causing a distal tip of the delivery cannula to follow a path formed by the non-straight predetermined shape. In some embodiments, the path is around the affected area. In some embodiments, methods can also include delivering the therapeutic agent at one or more regions along the path. In some embodiments, methods can also include monitoring electrical activity in or near the selected site using the delivery cannula.

Embodiments described herein can have one or more of the following advantages.

In some aspects, some of the systems and methods described herein can be implemented to deliver therapeutics to a wider range of targets within a tissue specimen (e.g., a brain) and reduce trauma of the tissue relative to some conventional systems. For example, using a pre-formed delivery cannula having a predefined shape can allow for delivering a therapeutic along a predefined three dimensional path (e.g., deflecting along at least two different planes). That is, a delivery cannula can be formed in a predefined shape that corresponds to a desired therapeutic delivery path based on the size and location of the injection target, structures around which the therapeutic is being delivered, as well as the type of therapeutic being delivered. For example, a delivery cannula may be formed in a predetermined shape so that, as the delivery cannula exits the guide cannula, the tip of the delivery cannula travels within or around a region of tissue to be treated without requiring additional external deflection forces. In this fashion, targets distant from, or lacking orientation with, the axis of the guide cannula can typically be reached. As a result of the predetermined delivery cannula design shape, a guide cannula can be inserted into tissue (e.g., a brain) and require fewer movements (e.g., placement, removal, adjustment, and re-insertion) while the delivery cannula reaches the desired target positions. For example, in some cases, a guide cannula could be inserted to one location and the delivery cannula can be deployed to deliver therapeutics to several targeted positions around a portion of the tissue (e.g., around a tumor) along the predetermined shape. Fewer movements and placements of the guide cannula can result in less trauma to the underlying tissue than could occur using a system in which the delivery cannula consistently exits its guide cannula in one orientation (e.g., at a consistent angle relative to the guide cannula). Further, because the diameter of the delivery cannula is smaller (e.g., significantly smaller) than conventional cannulas, more discrete and delicate structures can be targeted. Moreover, the reduced size of the delivery cannula further reduces trauma and collateral damage. Furthermore, because the delivery cannula does not require multiple reinsertions to achieve three-dimensional dissemination of therapeutic, surgical time can be reduced (e.g., significantly reduced), thus also reducing surgical risk and morbidity.

Additionally or alternatively, in some aspects, some of the systems and methods described herein can be implemented to deliver therapeutics in a more controlled manner than some conventional systems. For example, the delivery cannula described herein having a step taper region at its distal end, where a larger diameter surface forms a barrier to reflux, or backflow, of fluid therapeutic introduced through the distally reduced-diameter delivery cannula. This can help a therapeutic to be delivered more accurately and to permit the fluid therapeutic to be retained at the target site rather than escaping from the area of interest along the outer wall of the delivery cannula. The increased precision in delivery can help the therapeutic to act more efficiently at the site for which it was intended. Increased precision can result in enhanced performance for therapeutics with known efficacy, and it may augment validity for evaluations of novel therapeutics.

Additionally or alternatively, in some aspects, some of the systems and methods described herein can be implemented to help make therapy delivery systems simpler, require fewer components, and/or potentially easier to use and be more accurate than some conventional systems. For example, in some embodiments, forming the delivery cannula at least partially out of a conductive material (e.g., by forming the delivery cannula out of metal or by disposing a conductive portion (surface) along or within the delivery cannula) can reduce or eliminate the need for a separate electrode to be included in the delivery system or for electrophysiological mapping to be required prior to delivery of therapeutic. That is, electrodes can be used to measure or monitor electrical signals in a brain, such as areas of abnormal electrical activity, when the delivery cannula is inserted into the brain. Using conductive materials for the delivery cannula itself can make the delivery system more efficient to manufacture and easier to use than systems requiring an additional electrophysiologic apparatus.

In some aspects, the inventive concepts herein feature delivery systems and methods for delivering a therapeutic agent to a selected site, e.g., a desired location, in a subject. These systems and methods can allow for precise placement of selected amounts, e.g., very small (e.g., less than about 250, 200, 150, 100, 90, 80, 70, 60, 50, 40, 30, or 20 microliters) or large amounts, of a therapeutic agent to a predetermined site in a subject with minimal trauma to the subject. Use of the systems and methods herein to deliver a therapeutic agent to a subject can result in a level of tissue damage which is substantially less than that caused by known delivery devices. Moreover, the systems and methods herein can be used to disseminate numerous grafts in a three dimensional configuration within a subject with only a minimal number of penetrations into the subject. In addition, if the therapeutic agent to be delivered to the subject includes cells or tissue, the systems and methods herein can provide for increased survival of the cells or tissue in the subject. The systems and methods herein can also be used to remove, with great precision and minimal trauma to a subject, selected substances, cells, and/or tissues from a selected site in the subject.

Accordingly, systems for delivering a therapeutic agent to a selected site in a subject can include a guide cannula for penetrating a selected site in a subject to a predetermined depth and a delivery cannula for delivering the therapeutic agent to the subject. The guide cannula can have an axial bore extending therethrough which has an open proximal end and an opening at a distal portion thereof. The delivery cannula can have an axial bore extending therethrough, a flexible distal end portion, and an outer diameter which is less than the inner diameter of the guide cannula. The shape of the delivery cannula can enable the delivery cannula to be inserted within the bore of the guide cannula and also allows for movement of the delivery cannula along the bore of the guide cannula. The delivery cannula can be manufactured of an inert, e.g., nontoxic and nonreactive with host tissue and components thereof, material which can be formed into various shapes and sizes with selected specifications and which is flexible. As used herein, the term “flexible” refers to at least a portion, e.g., a distal portion, of the delivery cannula that is capable of being deformed or bent without breaking. The term “resilient” can refer to a portion of the delivery cannula being able to be bent or deformed by an external force being applied and return to its original shape when the external force is removed. The flexible portion of the delivery cannula can be capable of returning to its original position or form upon removal of a force which causes it to deform or bend. Typically, at least a portion of the delivery cannula can be deflected at an angle from the guide cannula to deliver the therapeutic agent to a selected site in a subject. The flexibility of the delivery cannula can allow for placement of a therapeutic agent in a three dimensional array in a subject with minimal trauma to the subject. The material from which the delivery cannula is produced can be flexible or pliable when formed into cannulas having very small diameters at their distal ends, e.g., from about 1 to about 200 micrometers, preferably from about 10 to about 190 micrometers, more preferably from about 20 to about 180 micrometers, yet more preferably from about 30 to about 170 micrometers, still more preferably from about 40 to about 160 micrometers, and most preferably from about 50 to about 100 to about 150 micrometers. The material can be manufactured from a variety of materials, such as glass, polymeric materials, e.g., polycarbonate, polypropylene, or other polymeric material described herein, and metals, e.g., stainless steel, shape memory alloys (e.g., nitinol), etc. In some embodiments, the delivery cannula can be manufactured of a glass, e.g., borosilicate, soda-lime glass. In some embodiments, the delivery cannula can be manufactured of silicon dioxide either in the form of fused quartz or fused silica. In some embodiments, the delivery cannula can be manufactured from more than one, e.g., a combination of the materials described herein. For example, the delivery cannula can be composed at its distal portion of the flexible material described herein and at its proximal portion of a more rigid material such as a metal, e.g., stainless steel.

In some embodiments, the luminal walls of the delivery cannula can be coated or covered with an anti-adhesive compound. Anti-adhesive compounds include compounds which inhibit or prevent adhesion of agents described herein, e.g., therapeutic agents or agents which excite or inhibit neurons, or components thereof, to the luminal wall of the delivery cannula. In some embodiments, an anti-adhesive compound is a silicon (e.g., silane, e.g., silane the substituent groups of which can be any combination of nonreactive, inorganically reactive, and organically reactive groups). In some embodiments, the anti-adhesive compound is a polymer (e.g., polyethylene glycol), peptide, protein (e.g., albumin, e.g., bovine serum albumin, gelatin), glycoprotein (e.g., anti-sticking factor-I (ASF-I, Roy and Majumder (1989)_Biochimica et Biophysica Acta 991(1): 114-122); anti-sticking factor II (ASF-II, Roy and Majumder (19 Feb. 2004) Journal of Cellular Biochemistry 44(4):265-274), polysaccharide, or lipid or a solution of any of the foregoing (e.g., serum, bovine serum, milk)).

Examples of polymers that can be used as anti-adhesive compounds or as components of the delivery or guide cannulas described herein include parylene (poly(p-xylylene)), acrylates including methyl acrylate, ethyl acrylate, propyl acrylate, n-butyl acrylate (BA), isobutyl acrylate, 2-ethyl acrylate, and t-butyl acrylate; methacrylates including ethyl methacrylate, n-butyl methacrylate, and isobutyl methacrylate; acrylonitriles; methacrylonitrile; vinyls including vinyl acetate, vinylversatate, vinylpropionate, vinylformamide, vinylacetamide, vinylpyridines, and vinylimidazole; aminoalkyls including aminoalkylacrylates, aminoalkylmethacrylates, and aminoalkyl(meth)acrylamides; styrenes; cellulose acetate phthalate; cellulose acetate succinate; hydroxypropylmethylcellulose phthalate; poly(D,L-lactide); poly(D,L-lactide-co-glycolide); poly(glycolide); poly(hydroxybutyrate); poly(alkylcarbonate); poly(orthoesters); polyesters; poly(hydroxy valeric acid); polydioxanone; poly(ethylene terephthalate); poly(malic acid); poly(tartronic acid); polyanhydrides; polyphosphazenes; poly(amino acids) and their copolymers (see generally, Svenson, S (ed.), Polymeric Drug Delivery: Volume I: Particulate Drug Carriers. 2006; ACS Symposium Series; Amiji, M. M (ed.)., Nanotechnology for Cancer Therapy 2007; Taylor & Francis Group, LLP; Nair et al. Prog. Polym. Sci. (2007) 32: 762-798); hydrophobic peptide-based polymers and copolymers based on poly(L-amino acids) (Lavasanifar, A., et al., Advanced Drug Delivery Reviews (2002) 54:169-190); poly(ethylene-vinyl acetate) (“EVA”) copolymers; silicone rubber; polyethylene; polypropylene; polydienes (polybutadiene, polyisoprene and hydrogenated forms of these polymers); maleic anhydride copolymers of vinyl methylether and other vinyl ethers; polyamides (nylon 6,6); polyurethane; poly(ester urethanes); poly(ether urethanes); and poly(ester-urea).

In some embodiments, the anti-adhesive compound can include a parylene (poly(p-xylylene)) coating.

The guide cannula is typically produced from an inert material which provides sufficient rigidity to stabilize the delivery cannula in the subject, e.g., which is stiff or rigid to such a degree as to be able to penetrate the subject such that at least its distal portion is adjacent to or in proximity to a selected site in the subject. In some embodiments, the guide cannula includes or comprises a metal, e.g., stainless steel, gold, and gold alloy, a glass, e.g., borosilicate, soda-lime glass, silicon dioxide either in the form of fused quartz or fused silica or other material that transmits light, or a plastic, e.g., a plastic comprising a polymer or other non-plastic polymeric material. In some embodiments, the delivery cannula includes or comprises a plastic, e.g., a polymer having a molecular weight of from about 10,000 to about 6,000,000 daltons, e.g., from about 10,000 to about 3,000,000 daltons, e.g., from about 10,000 to about 1,00,000 daltons, e.g., from about 10,000 to about 500,000 daltons. Examples of polymers that can be used in the guide cannula include synthetic rubber, bakelite, neoprene, nylon, polyvinyl chloride, polystyrene, polyethylene, polypropylene, polyacrylonitrile, polyvinyl butyral, silicone, and other polymers described herein.

In addition, the guide cannula can be manufactured from a combination of such materials.

In some embodiments, the distal end of the guide cannula can be a blunt end which reduces damage to the tissue of the subject upon insertion of the guide cannula into the subject. The distal opening of the guide cannula can be disposed at the distal end of the guide cannula, coaxial with the lumen thereof, or it can be a side wall mounted opening disposed in a side wall of the guide cannula. If the opening at the distal portion of the guide cannula is a side wall mounted opening disposed in a side wall, the side wall of the guide cannula opposite the side wall mounted opening can increase in thickness distally to converge with a distal aspect of the side wall mounted opening.

In some embodiments, the delivery cannula tapers from a point or location, e.g., a proximal portion, which is a selected distance from the distal end to form a tube having a diameter at its distal end which is smaller than the diameter at its proximal end. The delivery cannula can taper such that the distal end of the delivery cannula is at least about ten fold, preferably at least about 20 fold, more preferably at least about 50 fold, and most preferably at least about 100 fold or more smaller than the diameter of the proximal end of the delivery cannula. In some embodiments, the guide cannula has a diameter of about 0.5 millimeters to about 3 millimeters and the delivery cannula tapers from a point or location which is a selected distance from the distal end to a distal end to form a tube having a diameter at its distal end of about 1 micrometer to about 200 micrometers. In some embodiments, the delivery cannula includes or comprises a hinge mechanism which allows a first portion of the delivery cannula to move relative to a second portion of the delivery cannula such that the delivery cannula exits the guide cannula at a selected angle relative to the guide cannula, e.g., at a selected angle relative to the guide cannula, e.g., at an angle greater than 30 degrees relative to the guide cannula, e.g., greater than 35, 40, 45, 50, 55, 60, 65, 70, 75, 80, 85, or 90 degrees relative to the guide cannula. For example, the hinge mechanism can be placed at any portion (e.g., a distal portion which is located about 3, 2.5, 2, 1.5, 1, or 0.5 centimeter(s) from the distal opening of the delivery cannula) of the delivery cannula such that the second portion is able to move as it exits the guide relative to the guide cannula as described herein. In some embodiments, the delivery cannula includes or comprises a distal opening which can be at the distal end of the delivery cannula, coaxial with the lumen thereof, or in a side wall, e.g., a side wall opening.

The systems and methods described herein can further include or comprise means for moving the delivery cannula relative to the guide cannula, means for moving the guide cannula relative to the selected site in the subject (e.g., a motorized drive), means for aspirating and expelling the contents of the delivery cannula, means for supplementing the contents, e.g., therapeutic agent, of the delivery cannula while it remains in the subject, e.g., in the tissue of the subject, during a surgical procedure, means for recording electrophyisological events at the selected site in the subject (e.g., by using devices such as the Guideline 4000 LP+™ from FHC, Bowdoin, Me., and compatible software), means for detecting an obstruction in the delivery cannula, e.g, means for measuring pressure at the site in the subject, e.g., including use of pressure transducers such as strain gages, variable capacitor, and piezoelectric sensors, and/or means for transmitting selected wavelengths of light to the distal portion of the delivery cannula. In one embodiment, components of a stereotaxic apparatus provide the means for moving the delivery cannula relative to the guide cannula, the means for moving the guide cannula relative to the selected site in the subject, and the means for aspirating and expelling the contents of the delivery cannula. In some embodiments, the systems and methods herein can include means for locking or securing the delivery cannula in a selected position, e.g., a stationary position, such that the delivery cannula does not move, e.g., does not move in any axis (e.g., it is secured or locked such that it cannot be withdrawn, advanced, or rotated), during delivery of the agents described herein.

In some embodiments, the delivery cannula or the guide cannula is manufactured such that it includes a selected configuration of a material which has free electrons or charge carriers (“luminal material”), e.g., a metal (e.g., copper, silver, gold, palladium, platinum, iron, and ruthenium) along a side of a lumen, e.g., a strip of metal which can extend for a selected length of the delivery or guide cannula and which can have length, width, and thickness dimensions of from about 5 nanometers to 300 microns, e.g., from about 1 micron to about 300 microns, e.g., from about 5 microns to about 250 microns. In some embodiments, the luminal material strip, e.g., metal strip, in the delivery cannula or the guide cannula can extend the length of the cannula and have a width of about 10 microns and a thickness of about 2 microns. This luminal material coating, e.g., metal coating in the lumen of the guide or delivery cannula, allows for recording electrophysiological events at the selected site in the subject. In addition, such coatings allow for sensing of other conditions, e.g., impedance, temperature, at the selected site. In some embodiments, the delivery cannula or guide cannula is manufactured such that it includes a compound (e.g., thermosetting polymer, e.g., UV-curable epoxies, and solvent based polymers, e.g., polyurethane, polyimide, a ceramic) which provides structural support to the cannula. Example methods for manufacturing the delivery cannula or the guide cannula such that it includes a selected configuration of metal are known in the art, e.g., see manufacturing information from Optomec, St. Paul, Minn. and Albuquerque, N. Mex.

Therapeutic agents which can be delivered to a subject using the systems and the methods herein can include agents which have a therapeutic effect, e.g., reduce or eliminate deleterious symptoms or undesirable effects caused by, for example, disease or injury, and/or which preserve health, in a subject. The therapeutic agents can be delivered alone or in combination with a pharmaceutically acceptable carrier or diluent through the diameter of the delivery cannula to the selected site in the subject. Pharmaceutically acceptable carriers or diluents are art recognized formulations and include saline, aqueous buffer solutions, solvents and/or dispersion media. The use of such carriers and diluents is well known in the art. These carriers or diluents are preferably sterile and fluid to the extent that easy syringability exists. Preferably, the solution is stable under the conditions of manufacture and storage and preserved against the contaminating action of microorganisms. Such therapeutic agents include small molecules, toxins, lysed cell products, cells, e.g., neural cells, e.g., such as mesencephalic cells and striatal cells, glial cells, stem cells, e.g., stem cells which are precursors to neural or glial cells, and tissues, peptides or proteins (e.g., a microbial opsin, an antibody, a growth factor, e.g., a neurotrophic factor, e.g., a ciliary neurotrophic factor for treatment of amyotrophic lateral sclerosis, brain-derived neurotrophic factor for treatment of Parkinson's disease, glial growth factors for treatment of multiple sclerosis and Parkinson's disease, and a nerve growth factor for treatment of Alzheimer's disease), lipids, and viruses. These growth factors can be delivered to a subject together with cells or tissues using the delivery systems herein. The cells delivered to the subject using the delivery systems herein can be obtained from any source, e.g., mammals such as pigs, rodents, and primates, e.g., humans and monkeys.

Other examples of therapeutic agents include chemotherapeutic agents, e.g., small molecule or protein chemotherapeutic agents, which cross the blood brain barrier such as carmustine and chemotherapeutic agents, e.g., small molecule or protein chemotherapeutic agents, which do not cross the blood brain barrier such as cisplatin, photodynamic drugs or agents such as porphyrin analogues or derivatives, and antimicrobial agents such as antibiotics. In some embodiments, the chemotherapeutic agent is an anti-angiogenic agent. In some embodiments, the delivery systems herein can be used to deliver concentrated doses of chemotherapeutic agents directly to brain tumors, e.g., brain carcinomas, thereby bypassing systemic administration and its accompanying undesirable side effects. Similarly, the delivery systems herein can be used to deliver antibiotics to focal infectious processes in the brain of a subject, e.g., brain abscesses. Selected concentrations of these antibiotics can be locally administered using these systems without the limitation of the antibiotic's ability to cross the blood brain barrier. Photodynamic drugs or agents can be locally administered using the delivery systems herein, allowed to accumulate in precancerous or cancerous cells, and subsequently illuminated by light transmitted through the delivery cannula. Illumination of the cells containing the photodynamic drugs activates the drug which in turn results in destruction of the precancerous or cancerous cells.

Other therapeutic agents which are used to treat acute events such as trauma and cerebral ischemia, or agents which can be used to treat chronic pathological processes can also be delivered by employing the delivery systems herein. Examples of these agents include nitric oxide synthase inhibitors and superoxide dismutase to inhibit oxidative stress caused by trauma, ischemia, and neurodegenerative disease, thrombolytics, e.g., streptokinase, urokinase, for direct dissolution of intracerebral thrombosis, and angiogenic factors to help reestablish circulation to traumatized or infarcted areas.

Still other examples of therapeutic agents which can be delivered to a subject using the delivery systems herein include nucleic acids, e.g., nucleic acids alone, e.g., naked DNA, RNA (e.g., regulatory RNA, e.g., RNAi (e.g., siRNA, microRNA, antisense RNA) and nucleic acids, e.g., DNA or RNA in delivery vehicles such as plasmids, lipid (e.g., lipidoids) or lipoprotein delivery vehicles and viruses or particles, e.g., microparticles, e.g., nanoparticles (e.g., particles having a size in their greatest dimension of between about 10 nm to about 1000 nm)). For example, nucleic acids which can be delivered to a subject using the systems herein can encode foreign tissue antigens that cause tumors, e.g., brain carcinomas, to be attacked by the immune system. In addition, further examples of nucleic acids which can be delivered to a subject using the systems herein include nucleic acids which encode immunostimulators (e.g., cytokines, IL-2, IL-12, y-interferon) to boost the immune system, nucleic acids which encode antigens which render tumor cells more vulnerable or more susceptible to chemotherapy, e.g., Allovectin-7, and nucleic acids which encode apoptotic proteins which cause the tumor cells to self-destruct. Alternatively, nucleic acids encoding neurotrophic factors, deficient proteins, specialized receptors, et cetera can also be delivered to a subject using the delivery systems herein. Regulatory RNAs which can be delivered to a subject using the systems herein can target genes associated with neurodegenerative diseases, e.g., the huntingtin gene.

The therapeutic agents can be chronically infused into a subject using the delivery systems described herein. Chronic infusion can be accomplished by advancing the delivery cannula to the target site, e.g., target brain site, securing it to the surrounding bone structures, e.g., skull, with, for example, acrylic, and attaching a constant infusion device, such as a mini-osmotic pump loaded with the therapeutic agent to be infused or delivered.

In some embodiments, the delivery systems described herein can be used to deliver neural cells to a selected site, e.g., putamen, caudate, substantia nigra, nucleus accumbens, or hippocampus, in the central nervous system. For example, when neural cells, e.g., mesencephalic cells, are transplanted into subjects having Parkinson's disease, the cells are typically delivered to the putamen and caudate nucleus. In addition, neural cells, e.g., GABAergic neurons, can be delivered using the delivery systems herein to epileptic foci in the brain of a subject. Furthermore, the delivery systems herein can be used to deliver cortical neurons, e.g., hNT neurons, to repopulate areas of neurodegeneration caused by stroke or trauma.

The systems and methods herein can also feature methods for delivering a therapeutic agent to a selected site in a subject. Subjects who can be treated using this method include mammals, e.g., primates such as humans and monkeys, pigs, and rodents. Selected sites in a subject include locations to which it is desirable to deliver a therapeutic agent. Examples of such locations include areas of neurodegeneration in the central nervous system of a subject. These methods can include the steps of inserting a guide cannula having the features described herein such that its distal portion is proximal to a selected site in the subject and inserting a delivery cannula, which releasably holds a therapeutic agent, into the guide cannula. The delivery cannula can be inserted into the guide cannula a predetermined distance such that the distal end of the delivery cannula is proximal to an opening at the distal portion of the guide cannula. The methods can then include the steps of extending the delivery cannula through the opening at the distal portion of the guide cannula along a first extension path to the selected site in the subject, and releasing the therapeutic agent from the delivery cannula into the selected site in the subject to form an injection site. In some embodiments, the delivery cannula can be inserted into the guide cannula prior to insertion of the guide cannula into the subject. In some embodiments, the delivery cannula can be loaded with the therapeutic agent to be delivered to the subject after it is inserted into the guide cannula. The delivery cannula can taper from a point or location at a selected distance from a distal end to the distal end to form a tube having a diameter at its distal end which is smaller than the diameter at its proximal end.

In some embodiments, the method can further include, after the step of releasing the therapeutic agent to the selected site, the steps of retracting the delivery cannula a predetermined distance from the first injection site, and releasing, e.g., by injection, the therapeutic agent from the delivery cannula into a second selected site in the subject to form a second injection site. These additional steps can be repeated as desired, e.g., at least twice.

In some embodiments, the method also includes after the step of releasing the therapeutic agent to the selected site or a series of sites along one path, the steps of retracting the delivery cannula such that the distal end of the delivery cannula does not extend beyond the opening at the distal portion of the guide cannula, rotating the guide cannula a predetermined angle from the first extension path of the delivery cannula, extending the delivery cannula through the opening at the distal portion of the guide cannula along a second extension path to a second selected site or series of sites in the subject, and releasing the therapeutic agent from the delivery cannula into the second selected site in the subject to form a second injection site or sites. These additional steps can also be repeated as desired, e.g., at least twice. This method results in placement of transplants in a three dimensional configuration in the subject with minimal trauma to the tissues of the subject.

The systems and methods herein can also feature methods for testing or monitoring selected neuronal circuitry in a subject, e.g., a mammal, e.g., a primate such as a human, monkey, pig, or rodent. These methods can include the steps of inserting a guide cannula having the features described herein such that its distal portion is proximal to a selected site in the subject and inserting a delivery cannula, which releasably holds an agent that can excite or inhibit a neuron when exposed to light, e.g., a microbial opsin, (e.g., channelrhodopsins ChR2 and VChR1 to excite neurons, and halorhodopsin (NpHR), archaerhodopsin (Arch), and fungal opsins such as leptosphaeria maculansopsin (Mac) to inhibit neurons) into the guide cannula. The delivery cannula is inserted into the guide cannula a predetermined distance such that the distal end of the delivery cannula is proximal to an opening at the distal portion of the guide cannula. The methods can then include the steps of extending the delivery cannula through the opening at the distal portion of the guide cannula along a first extension path to the selected site in the subject, releasing the agent that can excite or inhibit a neuron from the delivery cannula into the selected site in the subject to form an injection site, delivering light to excite or inhibit the neurons, and then recording the activity, e.g., electrical activity, of the neurons. In some embodiments, the light is transmitted through either of the delivery cannula or the guide cannula. In some embodiments, the activity, e.g., electrical activity, of the neurons is measured using a means for electrophysiological recording. In some embodiments, the method further includes the step of administering a therapeutic agent at the site of neuronal activity, or a site in proximity thereto, e.g., within a centimeter of the site of neuronal activity, in order to assess its affect on the neuronal activity. In these methods, one or more delivery cannulas can be used to deliver the agent that can excite or inhibit a neuron when exposed to light and the therapeutic agent. In these methods, the system can include various additional means for accomplishing each step in the methods, e.g., the system can include means for moving the delivery cannula relative to the guide cannula, means for moving the guide cannula relative to the selected site in the subject (e.g., a motorized drive), means for aspirating and expelling the contents of the delivery cannula, means for supplementing the contents, e.g., therapeutic agent, of the delivery cannula while it remains in the subject, e.g., in the tissue of the subject, during a surgical procedure, means for recording electrophyisological events at the selected site in the subject (e.g., by using devices such as the Guideline 4000 LP+™ from FHC, Bowdoin, Me., and compatible software), means for detecting an obstruction in the delivery cannula, e.g., means for measuring pressure at the site in the subject, e.g., including use of pressure transducers such as strain gages, variable capacitor, and piezoelectric sensors, means for transmitting selected wavelengths of light to the distal portion of the delivery cannula, means for measuring the distance of extension of the delivery cannula from an opening, e.g., a side wall opening, in the guide cannula; and/or means for uncoupling the delivery cannula from the guide cannula, e.g., in order to remove the delivery cannula from the guide cannula.

In some embodiments, the delivery cannula is inserted into the guide cannula prior to insertion of the guide cannula into the subject. In some embodiments, the delivery cannula is loaded with the agent that can excite or inhibit a neuron when exposed to light to be delivered to the subject after it is inserted into the guide cannula. The delivery cannula can taper from a point or location at a selected distance from a distal end to the distal end to form a tube having a diameter at its distal end which is smaller than the diameter at its proximal end.

### BRIEF DESCRIPTION OF THE DRAWINGS

FIGS. 1A-1B depict schematic views of an example delivery system. FIG. 1A is an enlarged view of a delivery system in which the delivery cannula extends through a distal portion of the guide cannula. FIG. 1B is a perspective view of a delivery system together with an apparatus for manipulating the system.

FIGS. 2A-2D depict various example delivery cannulas for use in the delivery systems. FIGS. 2A and 2B depict the distal portion of an example delivery cannula. FIG. 2B is a close-up view of the tip of the delivery cannula. FIGS. 2C and 2D depict an alternative example embodiment in which the proximal end of the delivery cannula is replaced with a stainless steel cannula.

FIGS. 3A-3C depict intact and cut-away side views of an example delivery system.

FIGS. 4A-4D are cutaway sequential views of the distal portion of an example delivery cannula being extended from a guide cannula.

FIG. 5 depicts a diagram of an example stereotaxic device for use in a stereotaxic surgical procedure.

FIGS. 6A-6D depict the mechanics and geometry of an example delivery system and a three dimensional array of implants which can be placed at selected sites in a subject using the system.

FIGS. 7A-7C depict another example embodiment of a delivery system in which the delivery cannula is advanced along a single trajectory and along the same axis as the guide cannula.

FIG. 8 depicts another example embodiment of a delivery system in which the delivery cannula includes a hinge which allows it to the exit the guide cannula at a selected angle relative to the guide cannula.

FIG. 9 depicts another example embodiment of a delivery system in which the delivery cannula includes a side wall opening.

FIGS. 10A-10C are sequential side views of a delivery cannula extending from a side opening of a guide cannula in a pre-defined shape.

FIGS. 11A-11C are sequential side views of a delivery cannula extending from an open opening of a guide cannula in a pre-defined shape.

FIG. 12 is a side view of an example pre-defined shape of the delivery cannula.

FIG. 13 is a perspective view of an example pre-defined three dimensional shape of the delivery cannula.

FIGS. 14A-14D are sequential side cross-sectional views of a therapeutic delivery procedure using a delivery cannula having a predefined shape.

FIG. 15 is a side view of an example delivery cannula having a step-taper end.

FIG. 16 an enlarged side view of delivery cannula having a step-taper end.

FIG. 17 is an end view of the example step-taper.

FIG. 18 is a perspective view of an example step-taper.

FIG. 19 is a perspective view of an example step-taper having multiple step regions.

FIG. 20 is a perspective view of an example electrode disposed within a delivery cannula.

FIG. 21 is a perspective view of an example electrode applied along a delivery cannula.

### DETAILED DESCRIPTION

In the following detailed description, numerous specific details are set forth in order to provide a thorough understanding of example embodiments. It will be understood by those of ordinary skill in the art that these embodiments may be practiced without some of these specific example details provided. In other instances, known methods, procedures, components and structures may not have been described in detail so as not to obscure the embodiments of the systems and methods described herein. The contents of all cited references (including literature references, issued patents, published patent applications, and co-pending patent applications) cited throughout this application are hereby expressly incorporated by reference.

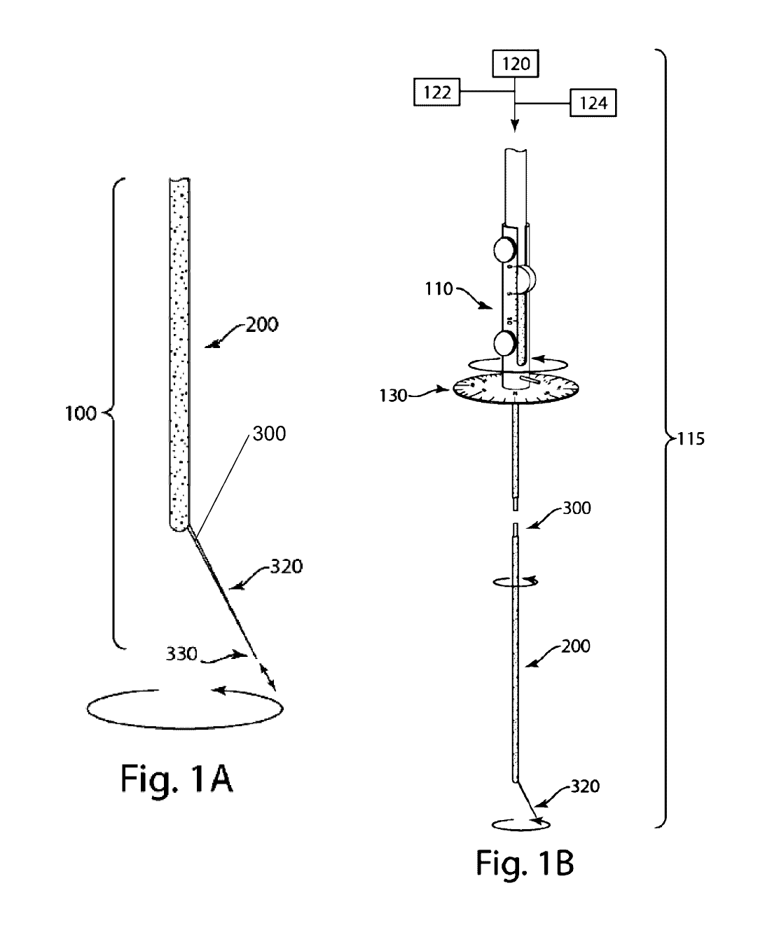

FIG. 1A illustrates an example delivery system (e.g., delivery catheter, delivery instrument, delivery apparatus) 100. FIG. 1B illustrates a delivery system combined with an apparatus for manipulating the system 115. The delivery system together with the apparatus for manipulating the system 115 includes a small-diameter guide cannula 200, e.g., a stainless steel guide cannula, a delivery cannula 300, including one or more of the delivery cannula features or properties described herein, configured to translate there within, a configuration of instruments for precise control of cannula depth, such as the vernier guide shown 110, means for aspirating and expelling 120 precise measurable volumes of the contents of the delivery cannula, such as a stylet or hydraulic mechanism, with a means for supplementing the contents of the delivery cannula while it remains in the tissue of the subject during a surgical procedure, means for recording electrophysiological activity 122, and means for transmitting light with predetermined wavelengths through the delivery cannula 124. The manipulation system can be mounted onto a standard stereotaxic instrument. An angle dial 130 can be used for precise control of rotation of the cannulas. Light delivery systems which can be used with the systems herein are commercially available from, for example, QLT, Vancouver, B.C. and PDT, Inc., Santa Barbara, Calif. Stereotaxic instruments which can be used with the systems herein are commercially available from, for example, Radionics, Inc., Burlington, Mass., and Westco Medical Corp., San Diego, Calif. Appropriate modifications of the delivery instrument manipulating devices, injection mechanisms, electrophysiological recording equipment, light delivery systems, and stereotaxic apparatuses are within the skill of the ordinary artisan. The delivery cannula 300 can be extended from the guide cannula to form a first extension path and then withdrawn into (or retracted within) the guide cannula 200. The guide cannula can then be rotated a predetermined angle within the subject and the delivery cannula extended from the guide cannula along a second extension path which is different from the first extension path.

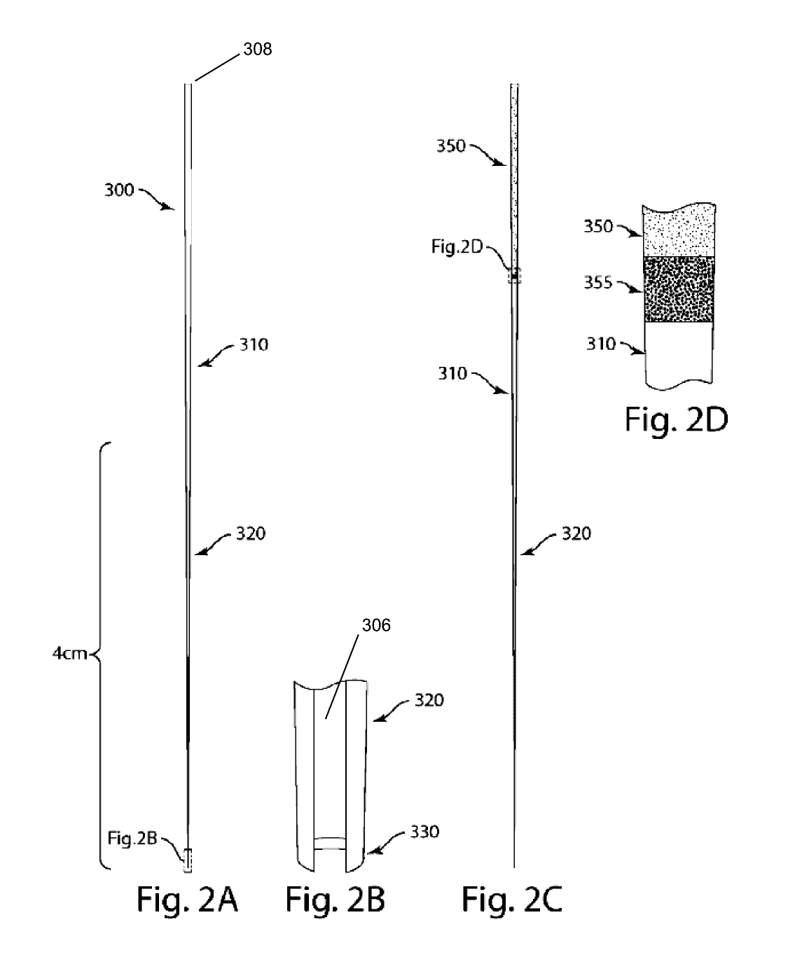

An example embodiment of the delivery cannula is illustrated in FIGS. 2A and 2B. In these figures, the delivery cannula 300 can be produced from (e.g., substantially or completely from) a long tube or pipette composed of, for example, glass, fused quartz or fused silica with an inner diameter (i.d.) of about 0.4 mm and outer diameter (o.d.) of about 0.7 mm. Such pipettes can be custom made of a variety of different materials in addition to glass, fused quartz, or fused silica and custom made to have a wide range of diameters. Using a modified glass electrode puller equipped with a lengthened heating coil and which is designed to accommodate a 10 cm or longer glass pipette, the pipette is pulled to produce a very long (about 4 cm) gently tapering shank 320. The delivery cannula tip 330, which is illustrated in FIG. 2B, is produced by removing the distal-most portion of the pulled pipette at an appropriate distance from the distal end to produce a delivery cannula with a selected distal end diameter. Any rough or sharp edges can be eliminated, i.e., smoothed out, by, for example, fire-polishing. Delivery cannula tips can be produced with diverse diameters to suit the properties of the therapeutic agent which is to be delivered to the subject. FIGS. 2C and 2D illustrate another example in which a metal cannula 350 of equal outer diameter as the delivery cannula, e.g., glass pipette, is substituted for at least a portion of the glass pipette and is affixed with epoxy or other suitable material 355 to the glass pipette 310 proximal to the beginning of the shank 320.

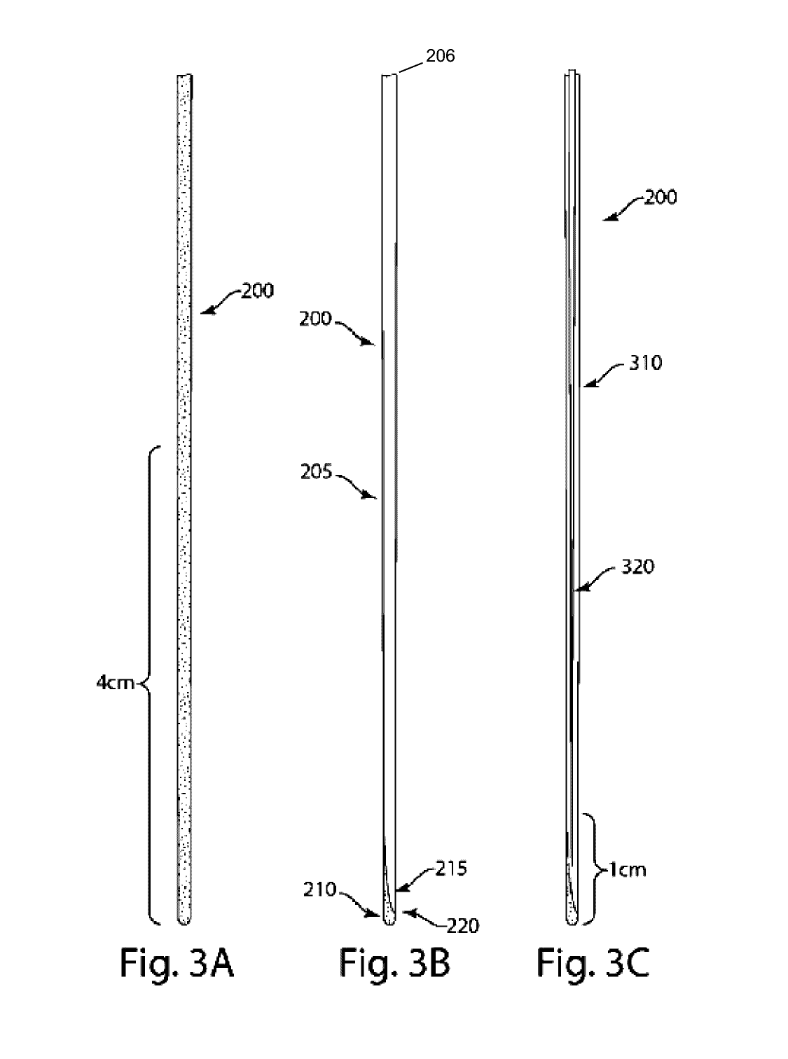

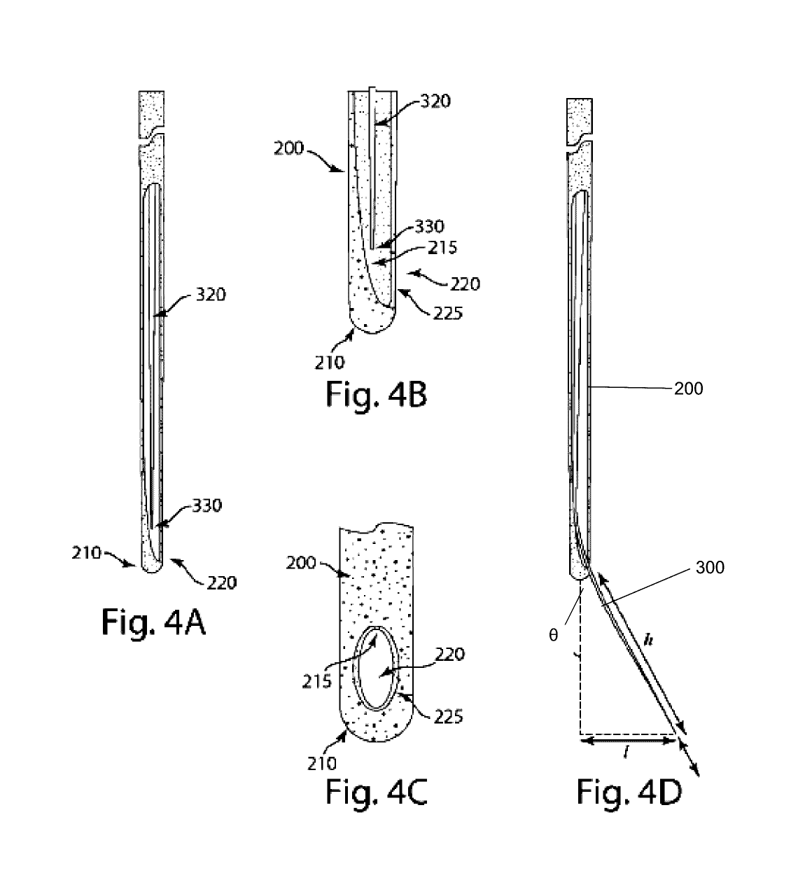

An example embodiment of the guide cannula is illustrated in FIGS. 3A-3C and 4A-4D. As illustrated, in some embodiments, the outer diameter of the guide cannula for delivery of a selected therapeutic agent to a selected site in a subject can be determined based on the following considerations: (1) the outer diameter should be a diameter which renders the guide cannula sufficiently rigid such that it is insertable into a subject without inadvertently deforming or bending (e.g., buckling) and such that it is rotatable in a subject with minimal deviation from its central axis, e.g., evenly rotatable (does not wobble or rotate unevenly from side to side); (2) the outer diameter should be minimized to the extent possible to reduce trauma to the subject upon insertion; and (3) the outer diameter should be a diameter which preserves an inner diameter which can accommodate a delivery cannula having a selected or desired outer diameter, e.g., having an inner diameter sufficient to allow delivery of a selected therapeutic agent to a selected site in a subject. The guide cannula 200 can be made from any of various structurally suitable and biocompatible materials. For example, some metals, such as stainless steel can be used. Alternatively or additionally, non-metallic materials, such as polymers, plastics, glass, quartz copolymers, ceramics, etc. can be used. Additional materials are described below, which may have other beneficial properties or performance characteristics.

With reference to FIGS. 3A-3C and 4A-4D, in some example embodiments, the guide cannula can be constructed from steel tubing (e.g., standard 19TW stainless steel tubing), with an outer diameter of about 1.07 mm and an inner diameter of about 0.8 mm, which permits passage of a delivery cannula with an outer diameter of about 0.7 mm. The length of the guide cannula 200 is typically sufficient to reach targets or selected sites in a subject at various distances with the use of a depth stop and with or without a conventional vernier guide for more precise depth placement.

The guide cannula 200 can include a distal end 210, a bore 205 passing therethrough, which can be used to guide the delivery cannula 300, and a distal opening (e.g., exit port) 220 that opens the bore 205 to the region outside the guide cannula 200 (e.g., the surrounding brain tissue). The distal end 210 of the guide cannula 200 can be blunt (e.g., rounded) so as to gently push tissue out of its path during penetration to thereby minimize trauma to the subject's tissue. The bore of the guide cannula 205 can be centrally located within the guide cannula 200 and extend throughout the length of the guide cannula 200 along the longitudinal axis of the cannula. The diameter of the bore 205 is typically greater than the maximum outer diameter of the uniform length 310 of the delivery cannula 300. In some cases, it can be beneficial for the delivery cannula 300 to extend from the guide cannula 200 from a side wall mounted opening, such as the distal opening 220, disposed in a side wall of the guide cannula 200. In such examples, one side of the distal inner wall of the guide cannula opposite the side wall mounted distal opening 215 typically increases in thickness distally (for example, for a length of about 0.5 to 1.0 cm) 215 to converge with a distal aspect of the side wall mounted opening. This increase in thickness of the side wall 215 opposite the side wall mounted distal opening 220 of the guide cannula bends or deflects the flexible delivery cannula 300 as the delivery cannula progresses downward within the bore of the guide cannula. By deflecting the delivery cannula 300, it can be directed in various parts of the tissue surrounding the guide cannula to deliver a therapeutic in the various locations desired, as depicted in FIG. 4D. This bend or curve in the delivery cannula 300 allows the delivery cannula to exit the guide cannula through the distal opening or exit port 220 just proximal to the distal end 210 of the guide cannula. The edges 225 of the distal opening or exit port 220 are typically smoothed or rounded to limit tissue damage or coring during penetration of the guide cannula. While the exit port 220 is generally described and illustrated as being formed along a side wall of the guide cannula 200, other configurations are possible. For example, in some embodiments, the exit port can be disposed at an end of the guide cannula.

In this manner and as shown in FIG. 4D, the delivery cannula is diverted in a manner dependent upon the characteristics of the thickness of the side wall opposite the distal opening and other factors such as the material from which the delivery cannula is manufactured, and the shaping and taper of the shank of the delivery cannula, and exits the guide cannula at a precise angle θ, thereafter traveling along a straight trajectory. The thickness of the side wall of the guide cannula opposite the distal opening 215 as well as any of the additional factors which contribute to the diversion of the delivery cannula can be modified to increase or decrease the exit angle θ of the delivery cannula. In addition, in an alternative embodiment, a groove or channel can be machined down the thickened wall 215 of the guide cannula, preferably down the center, to more accurately guide the distal portion or tip of the delivery cannula through the guide cannula to the selected opening or exit 220 at a distal portion of the guide cannula. Use of such a groove or indentation to guide the delivery cannula through the guide cannula minimizes side-to-side movement or motion of the delivery cannula during extension and retraction within the guide cannula. Referring to FIG. 4D, given the exit angle θ and the distance h, the distance from midline l can be calculated and the final target can be precisely reached.

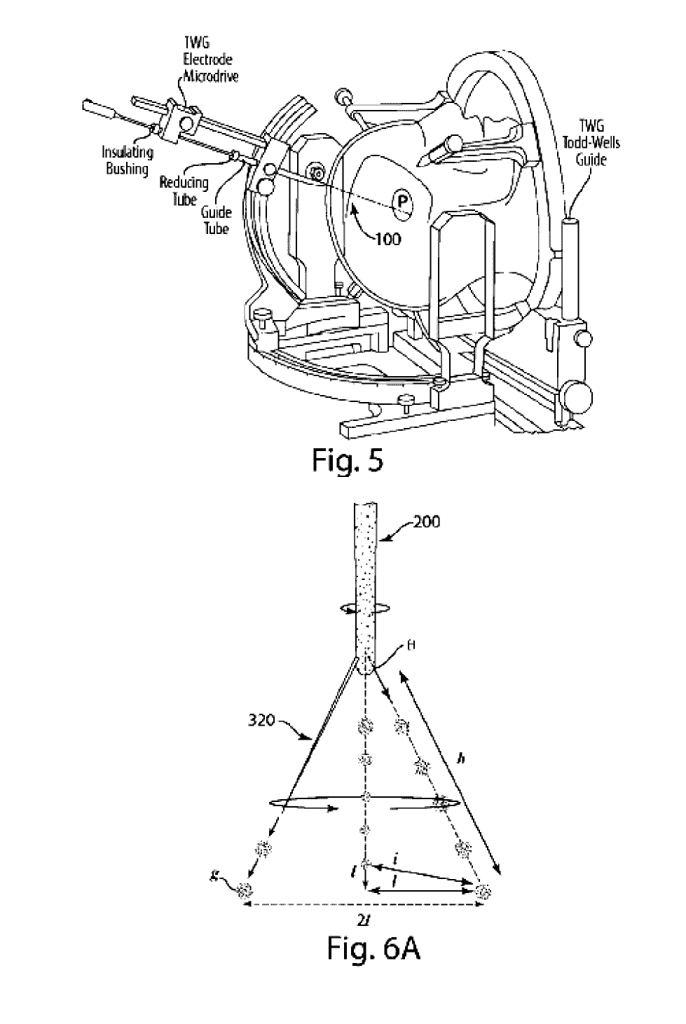

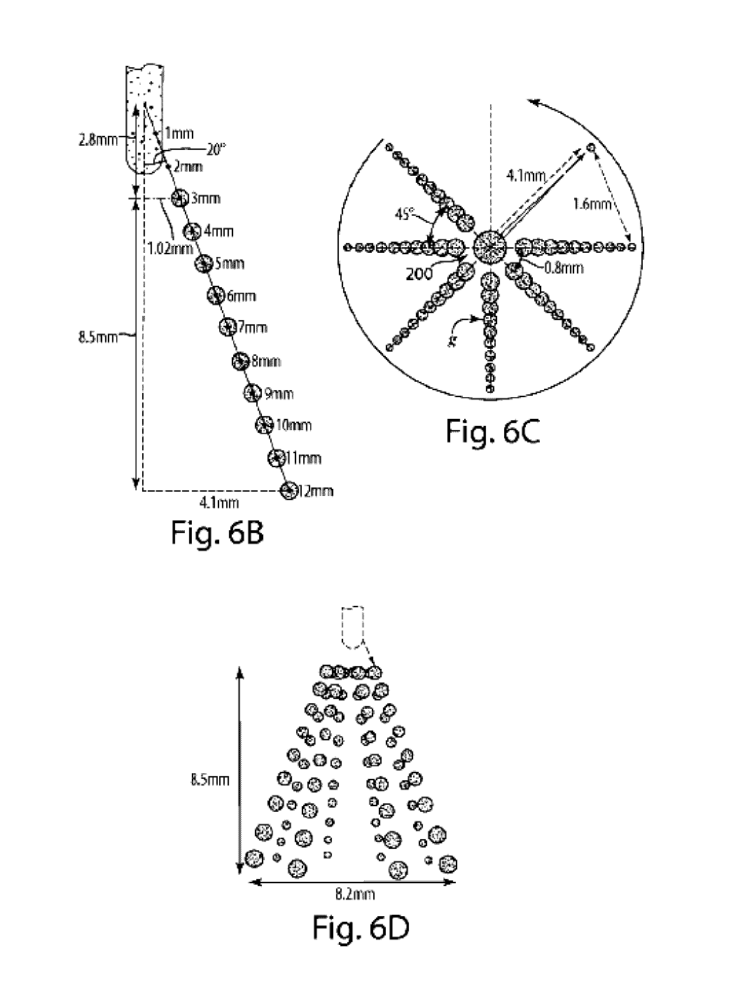

FIG. 5 depicts a stereotaxic apparatus which can be used in conjunction with the delivery systems described herein to deliver therapeutic agents to the brain, e.g., to the posterior putamen P, of a subject. These stereotaxic apparatuses are commercially available from Radionics, Burlington, Mass. FIG. 6A illustrates the procedure for distributing multiple injections of a therapeutic agent, such as neural cell grafts g, to a subject, in a three dimensional, e.g., conical, array. The delivery cannula is extended distance h from the end of the guide cannula at angle θ to form a first extension path. The distal-most injection is thus placed at distance l from the midline of the guide cannula. The diameter of the base of the array is thus 2×l. Withdrawal of the delivery cannula into the guide cannula can be interrupted at selected distances to allow numerous injections to be made along the trajectory of the delivery cannula to form a series of injections along the first extension path. Upon withdrawal of the delivery cannula into the bore of the guide cannula such that the distal end of the delivery cannula does not extend beyond the opening at the distal portion of the guide cannula, the guide cannula is rotated a predetermined angle from the first extension path of the delivery cannula and the delivery cannula is extended or advanced again through the opening at the distal portion of the guide cannula along a second extension path thereby allowing a new series of injections. Referring to FIG. 6A, the angle of rotation of the guide cannula determines the distance i between grafts of the first delivery cannula extension path and the second delivery cannula extension path and subsequent delivery cannula extension paths.

FIGS. 6B-6D are examples of scale diagrams of micrograft arrays as they appear in three-dimensional space. FIG. 6B illustrates a series of 10 implants of 0.5 microliters each which are placed 1 mm apart, along a single 12 mm delivery cannula trajectory, diverted from the guide cannula midline by 20°. If the therapeutic agent to be delivered includes cells, this implant volume need be spaced only every 0.5 mm to result in excellent survival and integration of the cells in the subject. To avoid the cellular and molecular mechanisms involved in tissue trauma and graft rejection, the implants delivered to the subject using the delivery systems herein are placed a selected distance from the distal end of the guide cannula, the source of the tissue trauma and the location of the deleterious cellular and molecular events contributing to graft rejection. Typically, the selected distance is about 1 mm from the distal end of the guide cannula. Thus, given the implant configuration illustrated in FIG. 6B, the graft furthest from the guide cannula is about 4.1 mm from the midline of the guide cannula, and the graft nearest the guide cannula is about 1.02 mm from the midline of the guide cannula.

FIG. 6C is a three-dimensional representation, viewed from the top, of the process of producing a micrograft array in which radial delivery cannula trajectories are at 45° angles. With this distribution, the centers of the grafts g most distal from the guide cannula are separated by about 1.6 mm, and the grafts most proximal to the guide cannula are separated by about 0.8 mm. FIG. 6D is a three-dimensional representation of the side view of a completed grafting array. The base of the conical array is about 8.2 mm across and its apex is about 1.02 mm across, while its height is about 8.5 mm. Thus, this configuration of 80 implants of 0.5 microliters each, 1 mm apart, disseminated from a single penetration of the guide cannula, allows for approximately 40 microliters of a therapeutic agent, e.g., cells, e.g., neural cells, to be implanted within a tissue volume in a subject of less than one cubic centimeter. The number of injections within a given area can be altered considerably depending on such variables as distance of delivery cannula extension, diversion angle of delivery cannula from the guide cannula, distance between injections, volume of injections, and angle of rotation between trajectories. Furthermore, these three dimensional arrays of implants can be stacked or tiered. These stacks or tiers are generated by injecting one array of implants of a therapeutic agent, withdrawing the guide cannula a selected distance, and repeating the injection procedure.

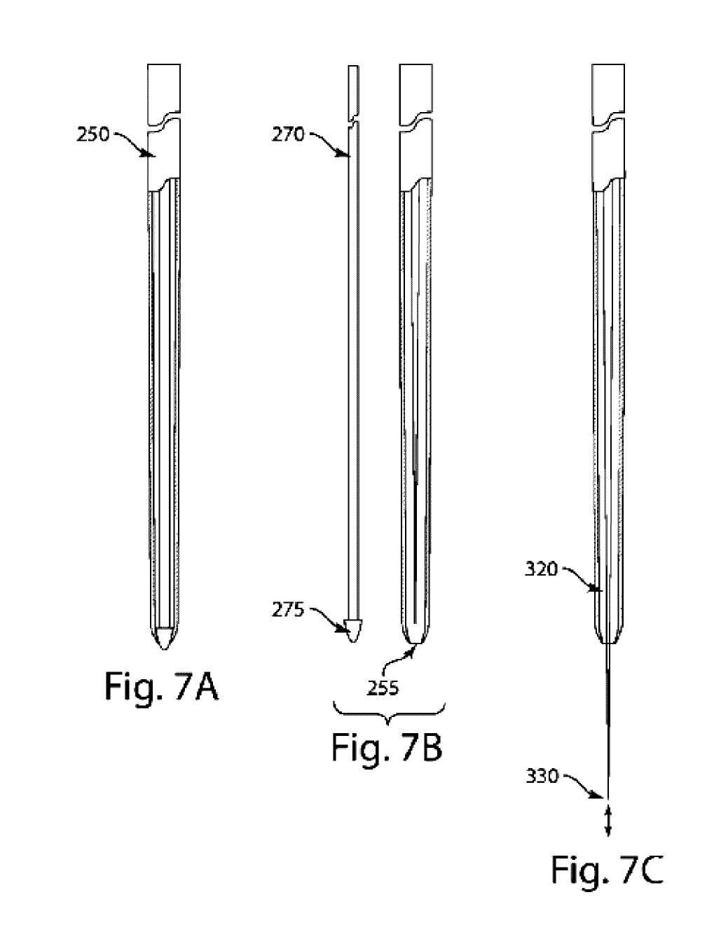

FIGS. 7A-7C illustrate another embodiment in which the guide cannula 250 is similar to the guide cannula 200 described above (see FIGS. 3A-3C and 4A-4D) except the bore is uniform for the length of the guide cannula and at the distal opening or exit port 255 at the end of the guide cannula it tapers circumferentially to accommodate the fitting of the blunt tip 275 of an occluder 270. With the occluder 270 in position, as in FIG. 7A, the end of the guide cannula is thus rounded and can be advanced into the subject, e.g., into the subject's brain, with minimal trauma to a point many millimeters proximal to the target. The occluder 270 is then removed and the delivery cannula 300 as described above (FIGS. 2A-2C) is extended or advanced through the guide cannula, and the tip 330 is extended from the distal opening or exit port 255 to the target. Similar to the procedure described above, withdrawal of the delivery cannula can be interrupted at specified distances to allow multiple injections to be made along the delivery cannula's trajectory. Alternatively, this simplified embodiment is suitable for single injections or for long-term infusion.

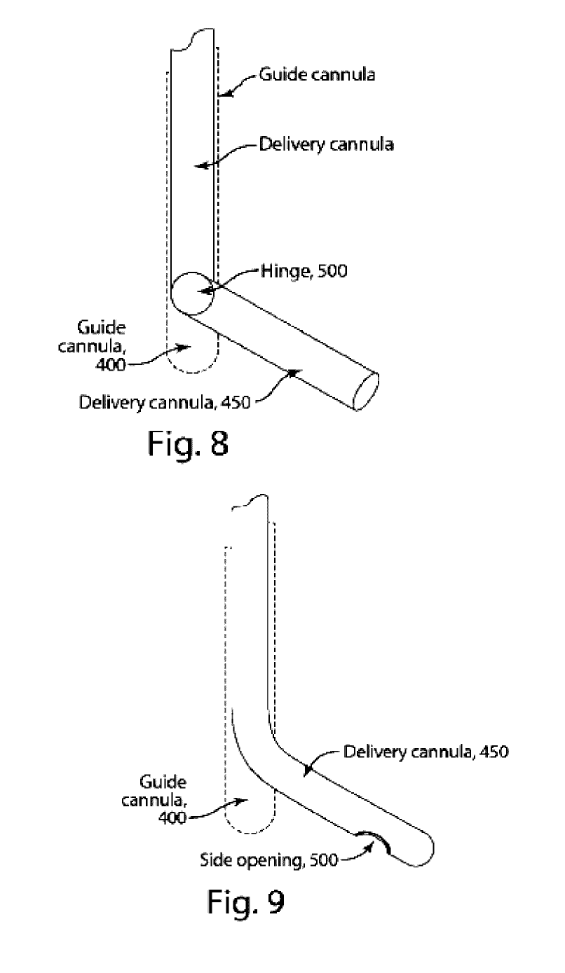

FIG. 8 illustrates another embodiment in which the delivery cannula 450 includes a hinge mechanism 500 which allows the delivery cannula to exit the guide cannula 400 at a selected angle relative to the guide cannula as described herein.

FIG. 9 illustrates another embodiment in which the delivery cannula 450 include a side wall opening 500.

In addition, the delivery cannula of the delivery systems herein can be guided through the guide cannula such that it bends and exits through an opening at the distal portion of the guide cannula at an angle to allow for approach of a selected target site while avoiding or bypassing important anatomical structures adjacent to and/or surrounding the site. Using the delivery systems herein, neural cells can be delivered to remote or high risk targets such as the substantia nigra with minimal inflammation and edema and with minimal risk of damaging important anatomical structures, e.g., the brain stem. Thus, the delivery systems or delivery apparatuses herein can be used to discretely and consistently place small volumes of a therapeutic agent at selected anatomical site(s) while preserving local cytoarchitecture. If cells are delivered using the delivery systems herein, cell survival in the subject can be increased two fold or more over that seen with the techniques presently used for human neural transplantation. In situations where it is desirable to use fetuses from humans or other mammals as a source of cells or tissue to be transplanted, this increase in cell survival using the delivery systems herein decreases the number of fetuses required to provide the same level of clinical improvement in the recipient subject. For example, if 10 fetuses from which cells are harvested for transplantation are normally required using the delivery devices in the art to produce a desired level of clinical improvement in a human, only 5 fetuses would be required using the delivery system herein to produce the same level of clinical improvement in a subject. The delivery systems or delivery apparatuses herein can also be used to deliver therapeutic agents, with minimal disruption, to spinal cord locations, peripheral nervous system locations and locations in and around, e.g., eye chambers, the eye, etc.

Additional applications of the delivery systems herein are diverse and include use in microbiopsy, electrophysiological recording, and photodynamic therapy. Just as tissue can be discretely placed in a selected site in a subject in one, two or three dimensional arrays, tissue can be removed from discrete, selected sites in a subject using the delivery systems herein in a one, two or three dimensional array. This is achieved by aspirating cells into the tip of the delivery cannula, or by first injecting a small volume of enzyme, such as trypsin, allowing a short incubation, and then aspirating the dissociated cells into the tip of the delivery cannula. In this embodiment, the delivery cannula becomes a removal cannula. Microbiopsies of aberrant cells, e.g., cancerous cells, using the systems herein can be performed with minimal trauma to the subject while reducing the risk of seeding, e.g., leaving a path of aberrant cells, normal tissue with aberrant cells. In addition, aberrant cells, e.g., cancer cells, can be removed using the systems herein, genetically manipulated in culture, and delivered to the subject as a vaccine with extremely high tumor specificity.

While the examples discussed above have generally described using the shape and structure of the guide cannula 200 as controlling or aiding in the deflection of the delivery cannula 300 and resulting curvature thereof, other techniques may be employed. For example, in some embodiments, the delivery cannula 300 can be pre-formed to be curved such that when extended from the guide cannula 200 it naturally deflects and follows a curved path (i.e., its pre-formed path). That is, an arc-shaped, pre-curved delivery cannula 300 can be manually straightened, for example, upon being inserted into the guide cannula. The manual straightening of the delivery cannula can cause it to be resiliently biased (e.g., deflected or bent from its free orientation with limited permanent deformation, but able to return to its free orientation once external forces are removed) in a straight orientation such that as the resisting force of the guide cannula's side wall is removed, for example, as the delivery cannula reaches the exit port 220, it can automatically curve without requiring external forces, such as those from the side wall of the guide cannula opposite the distal opening 215 discussed above. In some cases, as it exits the guide cannula, the delivery cannula may resiliently return to its curved shape that it followed prior to insertion into the guide cannula.

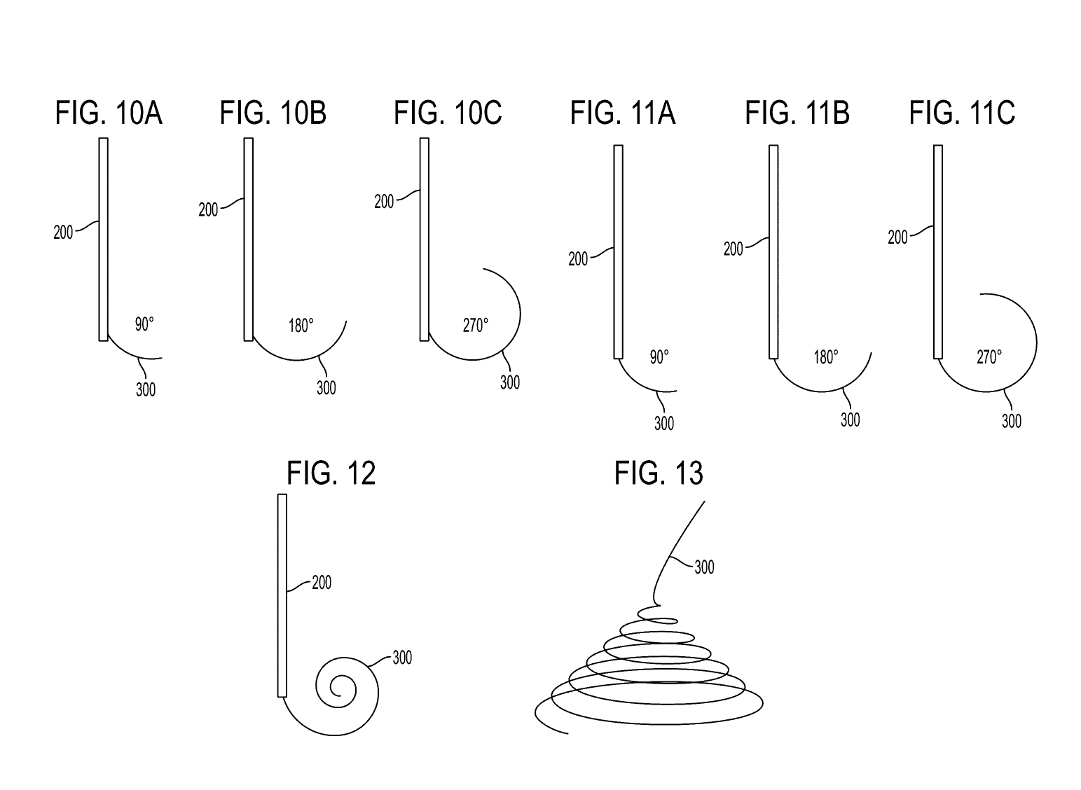

An example delivery cannula insertion sequence is depicted in FIGS. 10A-10C. In this example, a delivery cannula is shown, which has been formed in a predefined arcuate shape (e.g., circular). While it is within the guide cannula 200, the delivery cannula 300 is deflected (e.g., resiliently biased) to follow the generally straight path of the guide cannula 200. As illustrated, once extended from a side port of the guide cannula, the delivery cannula can arrange itself to resume to its predefined shape. As the delivery cannula 300 is deployed from the guide cannula 200 it will move along its predefined shape and range of angles, which can be any of various angles, e.g., at least 5°, 10°, 20°, 30°, 40°, 50°, 60°, 70°, 80°, 90°, 180°, 270°, etc. The diameter and angular bend of the arc of the predefined shape can be chosen based on the target area. For example, the delivery cannula may be pre-formed in a shape that can be used to provide therapeutics to multiple areas around a target. In some cases, the delivery cannula can be pre-shaped in a specific predetermined orientation so that as it exits the guide cannula, its tip may travel along a predetermined desired path around a specific predetermined structure, such as a fiber tract, ventricular space, vascular structure, tumor, or a portion of tissue affected by a medical incident (e.g., a stroke or arteriovenous malformation). In some cases, a user may shape the delivery cannula to deliver a therapeutic agent into the structure, for example, into a fiber tract.

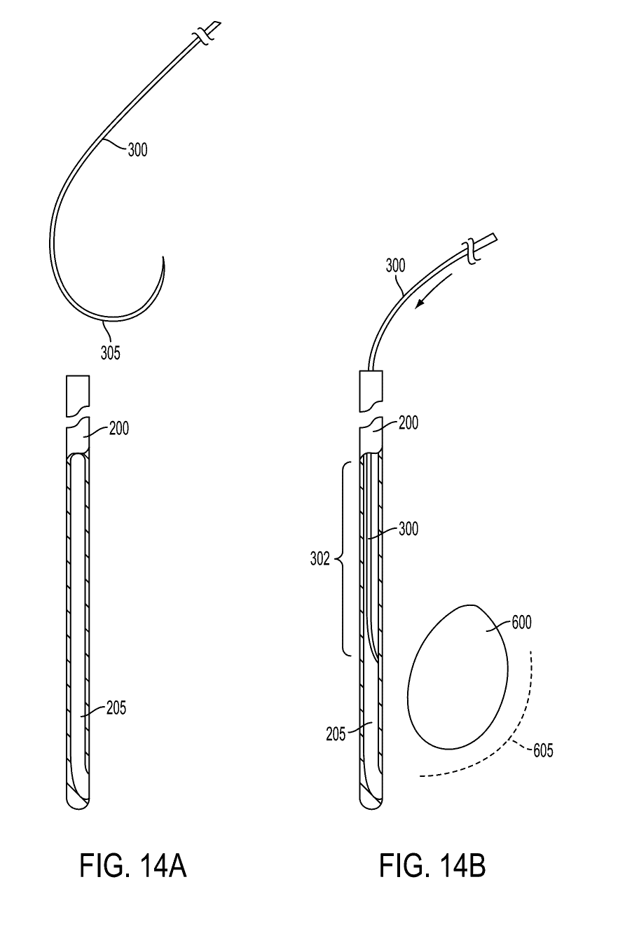

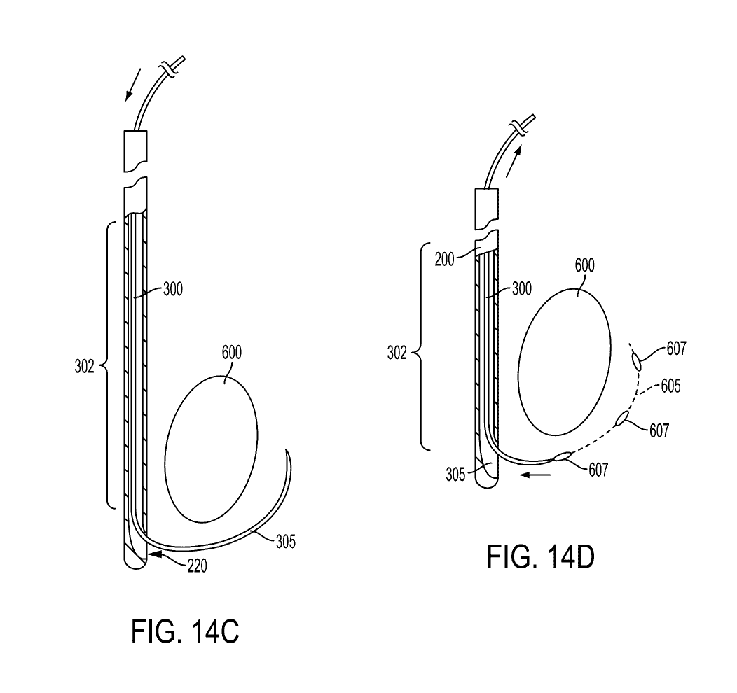

An example therapeutic agent delivery sequence is depicted in FIGS. 14A-14D. For example, a delivery cannula 300 can be formed to have a predetermined shape 305. As discussed herein, the predetermined shape 305 can correspond to a target area 600 of tissue to be treated. For example, the predetermined shape 305 can be substantially similar to a desired path 605 along which a therapeutic is to be delivered around the target area 600. Referring to FIG. 14B, the delivery cannula 300 can be inserted into a substantially rigid guide cannula 200. As a result of the substantially rigid guide cannula 200, a resiliently biased portion 302 of the delivery cannula can be temporarily straightened to conform to the shape of the bore 205 of the guide cannula 200 as it is inserted.

Referring to FIG. 14C, as the pre-formed delivery cannula 300 exits the guide cannula through the exit port 220, it can resume the predetermined shape 305. As illustrated, in some embodiments, the delivery cannula's predetermined shape 305 can be configured to position the delivery cannula around the target area 600. Referring to FIG. 14D, the therapeutic agent can be expelled from the delivery cannula at one or more regions 607 along the path 605 as the delivery cannula is either extended from the guide cannula or retracted within the guide cannula.

In this fashion, targets distant from the axis of the guide cannula can be reached. Advantageously, a therapeutic agent can be delivered to an array of targets in three-dimensional space by advancing the shape memory delivery cannula 300, injecting the therapeutic, retracting the delivery cannula 300, rotating the guide cannula and repeating the process. In some cases, the delivery cannula 300 need not be retracted into the guide cannula to reach multiple sites due to its predetermined shape.

The delivery cannula 300 can be formed of any of various types of materials that are capable of being pre-formed in a predetermined shape and remain resilient when deflected from the predetermined shape so that they return to, or substantially return to, the predetermined shape when an external deflecting force is released. In some cases, such materials can be referred to as a having a shape memory. In some embodiments, the delivery cannula 300 is made of a material with shape memory.

There are a number of materials that exhibit shape memory to return to their predetermined shape. In some embodiments, such shape memory materials can be metallic. These include shape memory alloys (SMA) including copper-aluminum-nickel and nickel-titanium (nitinol). Nitinol, for example, can be used in biomedical devices and exhibits shape memory and superelasticity and is biocompatible. These principles allow tubing composed of nitinol to be shaped (e.g., in an arc, a circular pattern, or another predetermined shape) at its transformational temperature (e.g., about 475° C.) for use as the delivery cannula 300. At normal temperatures, the tubing returns to its transformational shape after manipulation (e.g., straightening). Nitinol tubing with small diameters (e.g., about 50-300 microns) are amenable to this process. Thus, in some embodiments, a delivery cannula 300 with relatively small diameter, e.g., 50-300 microns, can be pre-shaped to assume, for example, an arcuate or circular shape, at the distal-most portion once extended from the guide cannula. Additionally, in some aspects in which shape memory alloys are used, electrical current can be applied to the delivery cannula to impart deflection of the delivery cannula.

Alternatively or additionally, other types of materials can exhibit shape memory to return to their predetermined shape. For example, certain plastic materials also demonstrate suitable shape memory so as to be possible alternatives. Known as “elastomers” or “shape memory polymers” (SMP), these materials are also suitable for the concepts described here. Examples of these materials include polyurethanes, polyethylene terephthalate (PET) and polyethyleneoxide (PEO). These materials are meant to be exemplary and not limiting.

While the examples described above with reference to FIGS. 10A-10C show and describe a side opening in the guide cannula, other embodiments are possible. For example, as shown in FIGS. 11A-11C, the delivery cannula 300 can be extended from a distal port of the guide cannula 200 and extend through its predefined path or shape. Unless otherwise described, features of the example in FIGS. 10A-10C can also apply to the example of FIGS. 11A-11C.

Additionally, while a circular or arcuate shape has been shown above, these are only examples, and the delivery cannula 300 can be formed in any of various other predetermined shapes. For example, referring to FIG. 12, the delivery cannula 300 can be pre-formed to have a spiral-shape sized and shaped to loop in on itself (e.g., forming one or more circular sections) as it is advanced out of the guide cannula. As illustrated in FIG. 13, the delivery cannula 300 can also be formed in any of various three-dimensional shapes, such as a substantially conical shape configured to deliver a therapeutic agent around a site. In some embodiments, the delivery cannula can be formed in a three-dimensional cork-screw type shape. In effect, the delivery cannula 300 could be pre-formed in a wide variety of predetermined three dimensional orientations, for example, in order deliver a therapeutic in tissue in a wide variety of predetermined patterns.

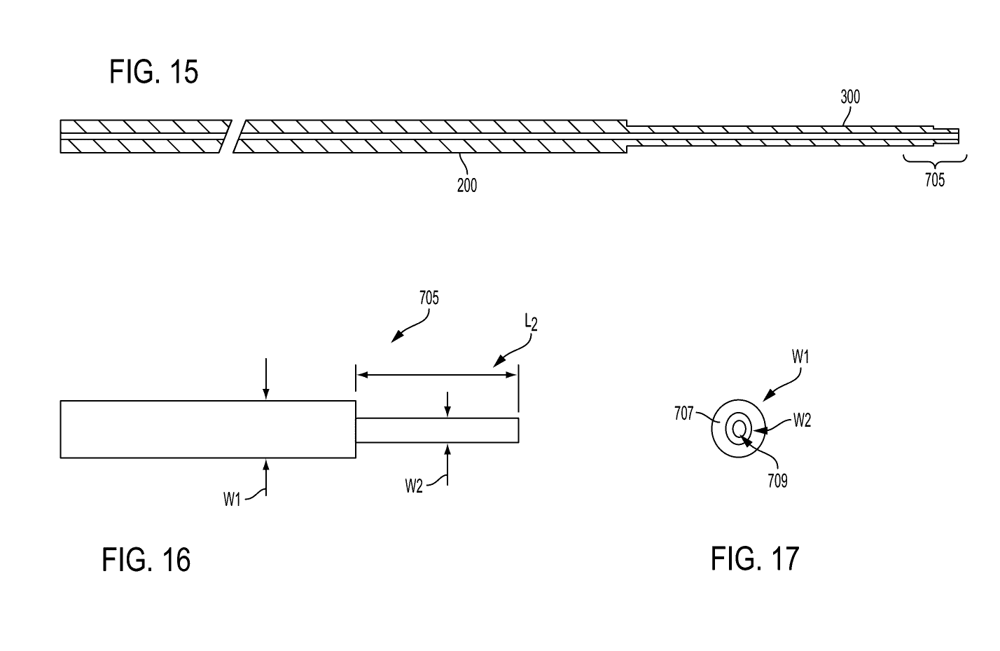

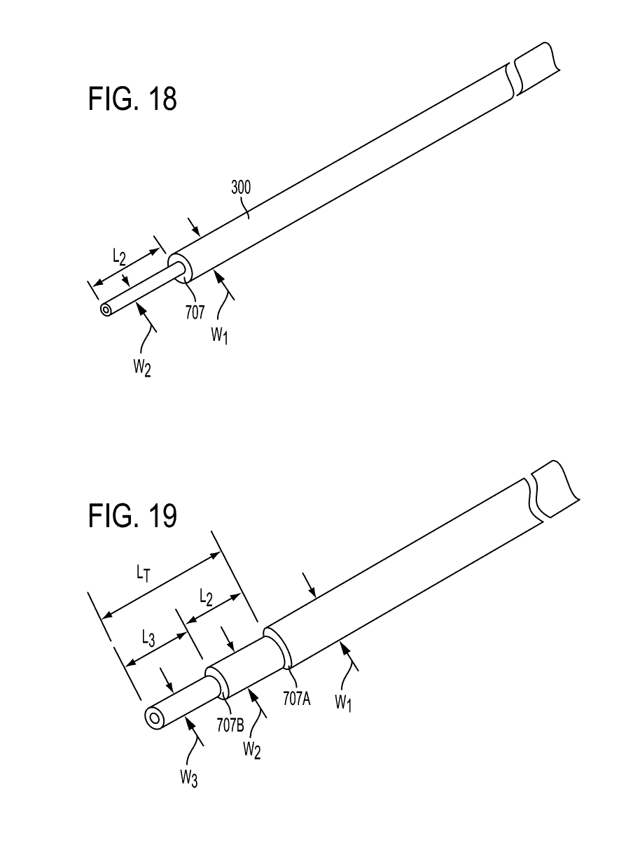

Implemented alone, or in combination with the various aspects described above, the delivery cannula 300 can have various other tip configurations. For example, referring to FIGS. 15-18, the delivery catheter 300 can include a step-down portion (e.g., step taper region) 705 at its distal end. For example, as illustrated in the enlarged view of FIG. 16, the tip of the delivery cannula can include a step where the width (e.g., diameter) transitions from a first region having a first width w1 to a second region having a reduced, smaller tip width w2 along a smaller tip length L2. The first width w1 can be the same as the average diameter of the delivery cannula (e.g., the diameter of the tubing from which the deliver cannula is formed).

The step-down portion 705 can span any of various lengths of the delivery cannula. For example, in some embodiments, the step-down portion 705 can be formed along the distal most 1-5 mm of the delivery cannula 300. In some cases, the smaller tip width w2 can be about 25% to about 75% (e.g., about 40% to about 60% (e.g., about 40%)) of the width of an adjacent region (e.g., the first width w1).

Advantageously, the step-down portion 705 can reduce backflow, also referred to as reflux, of fluid therapeutics, which can provide for better targeting and delivery of the therapeutic. For example, as illustrated in FIGS. 17 and 19, the difference between the first width w1 and the second width w2 can form a flow blocking surface 707 that helps to limit a therapeutic being expelled from the delivery cannula lumen 709 from flowing back proximally along the delivery cannula and away from the application site. Limiting this reflux can help yield a more accurate and controlled therapeutic delivery. That is, in some embodiments, the delivery systems described herein can be used to deliver a therapeutic to multiple locations around a region of tissue. Often, the precise placement and delivery of the therapeutic can help to increase the likelihood of success of the procedure. Therefore, limiting reflux using the step-down portion 705 can help to deliver a therapeutic into smaller, more discrete and precise locations.

For example, in some embodiments, the first width w1 can be about 10 microns to about 2000 microns (e.g. about 50 to about 400 microns). In some embodiments, the second width w2 can be about 5 microns to about 1000 microns (e.g., about 25 to about 200 microns). In some embodiments, the smaller tip length L2 can be about 100 to about 5000 microns (e.g., about 200 to about 2000 microns). In some cases, the first width w1 can be about 300 microns, the second width w2 can be about 100 microns, and the smaller tip length L2 can be about 1000 microns. In some embodiments, a ratio of the first width w1 to the second width w2 can be at least about 2:1 (e.g., at least about 3:1). In some embodiments, a ratio of the tip length L2 to the difference between the first width w1 and second width w2 can be greater than about 2.5:1 (e.g., about 5:1 to about 10:1). In some embodiments, a ratio of the flow blocking surface 707 to the cross sectional area of the second region having a diameter of the second width w2 can be at least 2:1 (e.g., about 5:1 to about 20:1).

While the examples illustrated and described with respect to FIGS. 15-18 relate to embodiments having one step (transitioning from a first width w1 to a second width w2), other embodiments are possible. For example, referring to FIG. 19, the delivery cannula can include more than one step. In some embodiments, the delivery cannula can include two, three, or more steps. In some embodiments, a delivery cannula can have two steps formed between a larger, outer width section having a diameter of first width w1, a middle width section having a diameter of second width w2 and a middle step tip length L2, and a smaller width section having a diameter of third width w3 and an end tip length L3. In some cases, a combined flow blocking surface can be formed of multiple surfaces, for example, as a combination of the end faces 707A, 707B of each of the steps. Additionally, a combined step tip length L_{T} can be formed of, for example, a combination of step lengths L1, L2.

Implemented alone or in combination with the various aspects described above, the delivery cannula 300 can be formed of one or more materials to permit omission of one or more other components from the delivery system 100. For example, as discussed in related application U.S. Ser. No. 13/699,464 by Cunningham, the delivery systems can also be used to record electrical, e.g., neural, activity, in a subject. For example, areas of abnormal electrical activity, e.g., epileptic foci, can be located using the delivery systems described herein. In this embodiment, the carrier of the therapeutic agent can include ions rendering the therapeutic solution electrolytic, which can permit the delivery cannula to serve as an electrode to receive the electrical activity. Once the site of abnormal electrical activity is located, the therapeutic agent can be delivered to the site also using one or more of the systems and methods described herein using standard electroencephalography. For example, because the therapeutic agent to be delivered can be in an electrolytic solution, recording and then delivery or injection can be achieved in a single step.

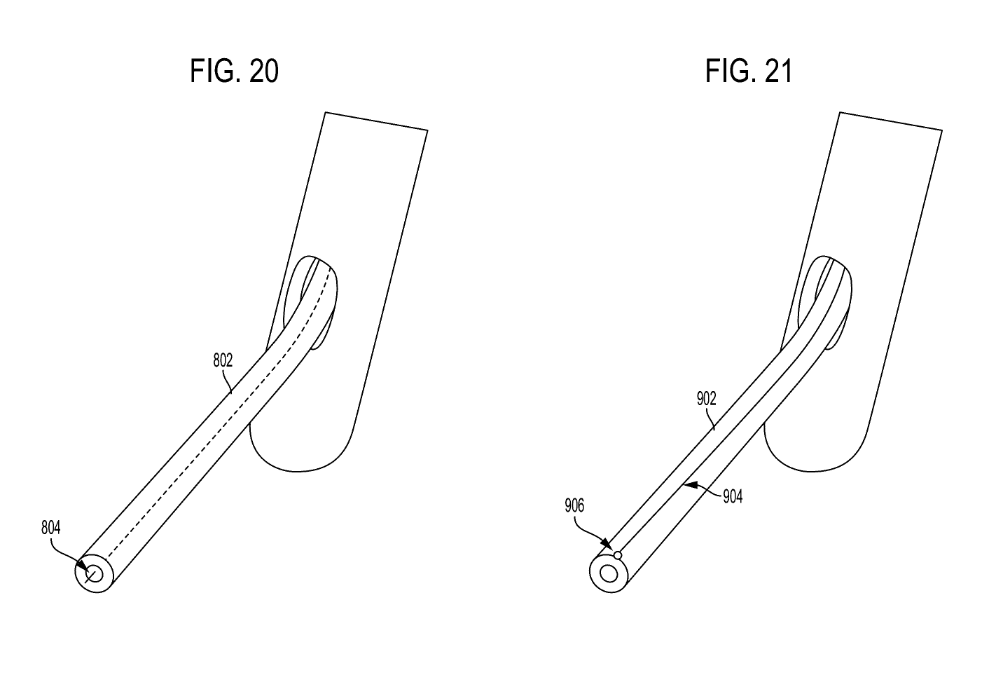

Additionally or alternatively, the delivery cannula itself can be formed, either partially or completely, of an electrically conductive material, such as a metal material (e.g., a shape memory alloy). For example, the delivery cannula can include a conductive portion forming an electrical circuit between a distal end of the delivery cannula and a proximal end of the delivery cannula. For example, referring to FIG. 20, in some embodiments, a conductive material (e.g., a wire or conductive strip) 804, can be disposed within the lumen of a delivery cannula 802. In some cases, disposing the wire within the lumen can electrically insulate the wire from surrounding tissue except for at its end (e.g., its distal end) so that it does not require additional insulation to separate is from tissue (e.g., brain tissue). Referring to FIG. 21, in some embodiments, the conductive material can be in the form of a conductive strip (e.g., a wire or an applied metallic trace) 904 along the outer surface of the delivery cannula 902. For example, the conductive strip 904 can be a metallic trace applied by a printing process (e.g., an inkjet application process). In some cases, the conductive strip 904 can be covered with an electrically insulating material so that a recording contact 906 is exposed at the tip of delivery cannula. The conductive material can be formed of any of various electrically conductive materials, such as metals (e.g., platinum, silver, or stainless steel).

Use of such electrically conductive material can allow for using the delivery cannula itself to detect and receive electrical activity. Using the delivery cannula as an electrode in this manner can help to make the delivery system simpler and easier to use by reducing the need for an additional wire disposed through the device (e.g., through the guide cannula).

Unless otherwise stated herein, example delivery cannula 802 and 902 can include one or more of the features, properties, or other aspects of delivery cannula 300 described herein.

An additional application for the delivery systems is in the field of photodynamic therapy for the destruction of cancer cells within precise foci. Photodynamic therapy is performed by injecting a photoreactive agent into a tumor site which preferentially accumulates within the tumor cells. With the delivery cannula still in position after delivery of the photoreactive agent, light is transmitted to the tip (distal portion) of the cannula (which can be designed to emit light) to thereby activate the photoreactive agent and destroy the tumor cells. Further description of methods of performing photodynamic therapy can be found in Fisher, A. M. et al. (1995) Lasers Surg. Med. 17(1):2-31 and Stables, G. I. et al. (1995) Cancer Treat. Rev. 21 (4):311-323.

### OTHER EMBODIMENTS

Having thus described several features of at least one embodiment of the present inventive concepts, it is to be appreciated that various alterations, modifications and improvements will readily occur to those skilled in the art. Such alterations, modifications and improvements are intended to be part of this disclosure and are intended to be within the scope of the systems and methods described herein. Accordingly, while various embodiments have been described herein, it should be understood that they have been presented and described by way of example only, and do not limit the claims presented herewith to any particular configurations or structural components. Thus, the breadth and scope of any embodiments or the claims should not be limited by any of the above-described exemplary structures or embodiments, but should be defined only in accordance with the following claims and their equivalents.

1. A system for delivering a therapeutic agent to a selected site in a subject, the system comprising:
a substantially rigid guide cannula defining an axial bore having an open proximal end and an opening near its distal end; and
a delivery cannula configured to fit within the guide cannula axial bore, the delivery cannula being pre-formed in a non-straight predetermined shape that differs from a shape of the guide cannula axial bore.

2. The system of claim 1 wherein the non-straight predetermined shape of the delivery cannula causes a portion of the delivery cannula disposed within the guide cannula to be resiliently biased to conform to the shape of the guide cannula axial bore.

3. The system of claim 2 wherein the portion of the delivery cannula disposed within the guide cannula is resiliently biased in a substantially straight orientation.

4. The system of claim 1 wherein a distal portion of the delivery cannula extending from the opening of the guide cannula resumes the non-straight predetermined shape.

5. The system of claim 1 wherein the non-straight predetermined shape comprises a curved profile.

6. The system of claim 1 wherein the non-straight predetermined shape comprises a three dimensional profile.

7. The system of claim 1 wherein the non-straight predetermined shape comprises a spiral shape.

8. The system of claim 1 wherein the non-straight predetermined shape comprises a bend of at least 5 degrees.

9. The system of claim 1 wherein the non-straight predetermined shape comprises at least 360 degrees of total bend angle.

10. The system of claim 9 wherein the at least 360 degrees of total bend are formed along a common plane.

11. The system of claim 1 wherein the non-straight predetermined shape corresponds to an identified structure to be treated by the therapeutic.

12. The system of claim 11 wherein the identified structure comprises a fiber tract.

13. The system of claim 11 wherein the identified structure comprises a portion of tissue affected by a medical incident.

14. The system of claim 1 wherein a distal portion of the delivery cannula comprises a step tapered region.

15. The system of claim 14 wherein a ratio of a width of a larger portion of the step tapered region to a width of a smaller portion of the step tapered region is at least about 2:1.

16. The system of claim 1 wherein the delivery cannula comprises a conductive portion forming an electrical circuit between a distal end of the delivery cannula and a proximal end of the delivery cannula.

17. The system of claim 16 further comprising an insulating material disposed over a portion of the conductive portion.

18. The system of claim 16 wherein the conductive portion comprises the delivery cannula being formed of a shape memory alloy.

19. A method of delivering a therapeutic agent to a selected site in a subject, the method comprising:
identifying a geometric property of an affected area to be treated with the therapeutic agent;
causing formation of a non-straight predetermined shape in the delivery cannula, the non-straight predetermined shape being based on the geometric property of the affected area; and
inserting the delivery cannula having the non-straight predetermined shape into a substantially rigid guide cannula defining an axial bore having an open proximal end and an opening near its distal end.

20. The method of claim 19 further comprising upon insertion of the delivery cannula into the guide cannula, resiliently biasing a portion of the delivery cannula disposed within the guide cannula to conform to the guide cannula axial bore.

21. The method of claim 19 further comprising inserting the delivery cannula further into guide cannula thereby causing a distal tip of the delivery cannula to follow a path formed by the non-straight predetermined shape.

22. The method of claim 21 wherein the path is around the affected area.

23. The method of claim 21 further comprising delivering the therapeutic agent at one or more regions along the path.

24. The method of claim 19 further comprising monitoring electrical activity in or near the selected site using the delivery cannula.
